(19) United States
(12) Patent Application Publication
(10) Pub. No.: US 2010/0280608 A1
(43) Pub. Date: Nov. 4, 2010

(54) INTRAOCULAR LENS

(75) Inventors: Wilhelm Stork, Impflingen (DE); Joergen Petersen, Goettingen (DE)

Correspondence Address: SEED INTELLECTUAL PROPERTY LAW GROUP PLLC, 701 FIFTH AVE, SUITE 5400, SEATTLE, WA, 98104, US

(73) Assignee: ACRI.TECH AG GESELLSCHAFT FUERO OPHTHALMOLOGISCHE PRODUKTE, Hennigsdorf (DE)

(21) Appl. No.: 11/909,608

(22) PCT Filed: Oct. 26, 2005
(86) PCT No.: PCT/EP2005/011473

§ 371 Date: Jul. 16, 2010

(30) Foreign Application Priority Data
Mar. 24, 2005 (DE) 10 2005 013 877.2
May 20, 2005 (DE) 10 2005 023 480.1

(51) Int. Cl. A61F 2/16

(52) U.S. Cl. 623/6.27

(57) ABSTRACT

The invention concerns an intraocular lens comprising an optical lens part which has a rear face (12, 22, 42, 52, 62) which can be towards the retina, wherein at least one optically effective face portion of the rear face has an at least approximately spherical curvature configuration, whose radius of curvature corresponds to the spacing of the face portion with respect to the retina in the region of greatest visual acuity.

The invention concerns an intraocular lens. Such a lens is known for example from EP 1 185 220 B1 to the same proprietor.

Intraocular lenses are generally composed of an optical lens part and a peripherally adjoining haptic part for fixing the intraocular lens in the eye. The imaging properties of the optical lens part are adjusted to the conditions of the eye to be treated. In that respect it is possible to use both lenses having purely refractive properties and also lenses having superposed diffractive properties. By way of example EP 1 185 220 B1 presents a lens which on the one hand refractively focuses the light rays in a central lens region, wherein superposed on the same lens region is a diffractive fine structure which produces at least one second focus. With such a lens the patient can see sharply both at distance and also close up.

It is further known for such lenses to be subdivided in an edge region into concentric annular zones which are displaced in the direction of the optical axis in such a way that the difference in path length of the light ray path at the transition of respectively adjacent zones is an integral multiple of the design wavelength of the lens. That lens configuration provides that a lens can be substantially thinner while retaining its refractive power and while being of the same optical area. Such a lens is particularly suitable for minimally invasive interventions on the eye as it can be folded together well, even when relatively high refractive powers are involved.

Particularly in connection with a vitrectomy in which the vitreous humor in the interior of the eye is removed however the problem which arises is that generally the imaging properties of the lens disposed in front thereof change. That is related to the different refractive indices of the tamponades which are introduced in that operation, or the substitution medium for the vitreous humor. By way of example water, gas or silicone oil fillings are known. Mixed substances based on partially fluorinated alkanes and oil are also used, which have respectively different refractive indices.

Therefore the object of the invention is to provide an intraocular lens in which aberrations are as small as possible irrespective of the substitution medium introduced.

That object is attained by an intraocular lens as set forth in independent claims 1, 11 and 15.

The intraocular lens according to the invention, in the region of its optical lens part, along the rear face which is towards the retina, has at least one optically effective face portion with an at least approximately spherical configuration, the radius of curvature of which corresponds to the spacing of the face portion with respect to the retina in the region of greatest visual acuity.

The intraocular lens can be implanted at various positions in the eye, for example as an anterior chamber lens, that is to say between the cornea and the iris, or as a posterior chamber lens, that is to say behind the iris and in front of the vitreous humor, mostly in the capsule sac of the natural lens. In addition the spacing with respect to the retina varies in dependence on the size and shape of the eyeball. The imaging properties of the lens are set thereto having regard to the overall optical system, cornea, possible anterior chamber lens and intraocular lens, and the curvature of the rear face is also correspondingly adapted to the imaging properties of the front face.

In accordance with the invention in that respect the curvatures of the face portions are at least approximately spherical, that is to say in the ideal case they are actually spherical or for example parabolic so that the deviation from the shape of a spherical surface is negligible at least in the proximity of the center of the face portion. The fact that the center of curvature of the concave face portions is on the retina and indeed in the region of greatest visual acuity, that is to say in the macula or the fovea, provides that the wave front which is incident at the rear face after refraction and/or diffraction at the front face is not additionally refracted there independently of the refractive index of the lens and the medium therebehind.

In other words, in the case of a lens according to the invention which is set in that fashion, light incident parallel to the optical axis is refracted and/or diffracted exclusively upon entry by way of the front face thereof into a beam focused on to the retina. That beam passes through the rear face without refraction once again as it is always perpendicular to the rear face of the lens. That ensures that the geometrical spacing between the intraocular lens and the focal point of the overall optical system always remains the same irrespective of the tamponade or the substitution medium.

In that respect minor deviations from sphericity produce only a slight degree of refraction at the rear face and thus minor aberrations due to the displacement of the focal point. Slight asphericity can even be provided for the correction of aberrations of the front face and the overall system. In regard to the configuration of the rear face however it is to be noted that the curvature thereof is approximated to the spherical shape to such an extent that, irrespective of the refractive index of the tamponade used or the vitreous humor, the aberrations are not disturbingly noticed by the patient, due to refraction at the transition from the rear face of the lens to the volume of the vitreous humor.

In an advantageous configuration the radius of curvature is between 12 mm and 30 mm and particularly preferably between 13 mm and 23 mm. That range covers most spacings which occur according to experience in respect of the intraocular lens from the retina, in dependence on the place of implantation and the shape and size of the eyeball.

Preferably a first optically effective face portion with an at least approximately spherical curvature configuration is provided in a central, purely refractive lens region of the rear face. That face portion may cover only a part or the entire optically effective rear face.

It is further advantageous if at least one second optically effective face portion with an at least approximately spherical curvature configuration is provided in a diffractive lens region of the rear face, surrounding the central lens region, concentrically adjoining the first optically effective face portion, wherein the radius of curvature of the second optically effective face portion corresponds to the spacing thereof with respect to the retina in the region of greatest visual acuity. Preferably in that respect the difference in path length of the ray path at the transition from the first optically effective face portion to the second optically effective face portion is an integral multiple of n>1 of the design wavelength.

In an advantageous configuration further optically effective face portions with an at least approximately spherical curvature configuration are provided in the annular diffractive lens region concentrically adjoining the second optically effective face portion and also each other, wherein the radius of curvature of the further optically effective face portions respectively corresponds to the spacing thereof with respect to the retina in the region of greatest visual acuity. Preferably in this case also the difference in path length of the ray path in the transition of the respectively adjacent optically effective face portions is an integral multiple of n>1 of the design wavelength.

In these embodiments the rear face is subdivided into a plurality of face portions forming annular concentric zones. That lens configuration provides that a lens can be substantially thinner while retaining the optical properties, in particular the geometrical spacing between the intraocular lens and the focal point of the overall optical system, irrespective of the tamponade or the substitution medium, with the same optical area, and is therefore particularly suitable for minimally invasive interventions on the eye.

In a preferred embodiment retaining the differences in path length between two respective adjacent face portions or zones is achieved by a geometrical step height h in parallel relationship with the ray configuration, that is to say the difference in the radii of curvature of two adjacent face portions/zones, which satisfies the following equation:

h·(n_{IOL}−n_{tamponade1})·N=h·(n_{IOL}−n_{tamponade2})·M,

wherein N and M are whole numbers and n_{IOL}, n_{tamponade1} and n_{tamponade2} are the refractive indices of the intraocular lens and two different given tamponades in the vitreous humor of the eye.

In general the rear face can therefore be purely refractive or mixed refractive-diffractive. In that respect the term refractive is used to denote an approximately spherical face (face portion) which at least for rays which are incident in axis-parallel relationship, in the ideal case, produce precisely no additional refraction. For rays which are incident at an angle to the optical axis they are admittedly nonetheless also refracted at the rear face, but at least less than in the case of known intraocular lenses and substantially only with the effect of a change in the size of the imaging with different tamponades or substitution media. In that respect the important consideration is that, in the sum of the optical properties of the rear face (refractive and/or diffractive) it does not cause any change in the wave front, at least for the axial beam.

Preferably the optically effective face portions are of a sawtooth configuration.

Preferably the central lens region is of a diameter of about 4 mm.

In a further preferred feature the optical lens part overall, that is to say also on the front face, has a central lens region and an annular lens region surrounding same, with a common focus. In that case the annular lens region has at least one first concentric annular zone, wherein the difference in path length of the ray path at the transition from the central lens region to the first annular zone and the transition of further, respectively adjacent annular zones, is an integral multiple of n>1 of the design wavelength.

In an advantageous development a diffractive fine structure extends at least portion-wise over the front face to form a multifocal, in particular bifocal, lens. In general terms it is sufficient if the diffractive fine structure only extends over the central lens region as in particular the bifocal function is only required in relation to a level of brightness corresponding to daylight, and the pupil opening of the eye is opened substantially only in the region of the central lens region. The additional diffractive fine structure can be in particular in the form of concentric zones or (sub-)face portions arranged around the optical lens axis. The difference in path length of the ray path in the transition of respectively adjacent zones or (sub-)face portions of the diffractive fine structure on the front face is in the case of a bifocal lens a fraction of the design wavelength, preferably alternately between 0.5 and 0.8 times and between 0.5 and 0.2 times and particularly preferably 0.4 and 0.6 times respectively the design wavelength.

By virtue of the concave rear face of the intraocular lens the front face must have a higher refractive power and thus curvature. As a result the lens is of a greater mean thickness than a biconvex lens. In order to counteract that the lens is advantageously in the form of a diffractive-refractive lens. Because a smaller mean thickness is achieved with the same refractive power, that preferred embodiment combines the advantageous imaging properties of the intraocular lens according to the invention with the advantage of a flat design so that it can be folded for the purposes of implantation and thus permits minimally invasive intervention. The difference in path length of the adjacent zones can be set on the one hand by the refractive index or by a suitable choice of material and/or on the other hand by the geometry of the respective zone. The annular zones are in that case advantageously of a sawtooth-like configuration in cross-section.

Further features and advantages of the invention will now be described in greater detail by means of embodiments with reference to the drawings in which:

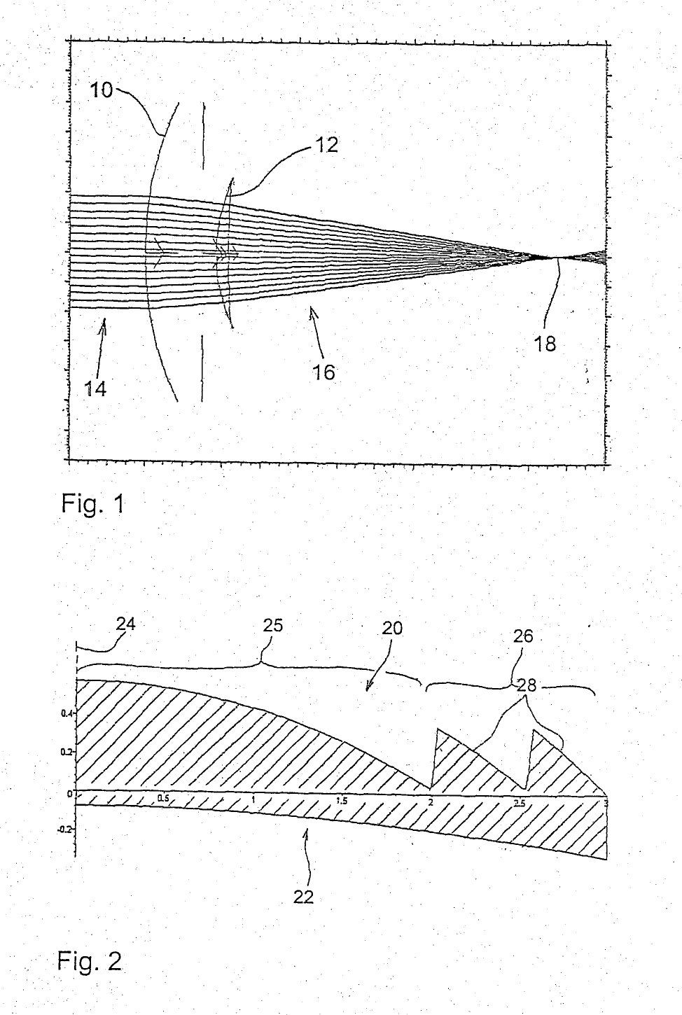

FIG. 1 is a diagrammatic view of the ray path through the eye with an intraocular lens according to the invention,

FIG. 2 shows a sectional view through one half of a lens body of the intraocular lens according to the invention,

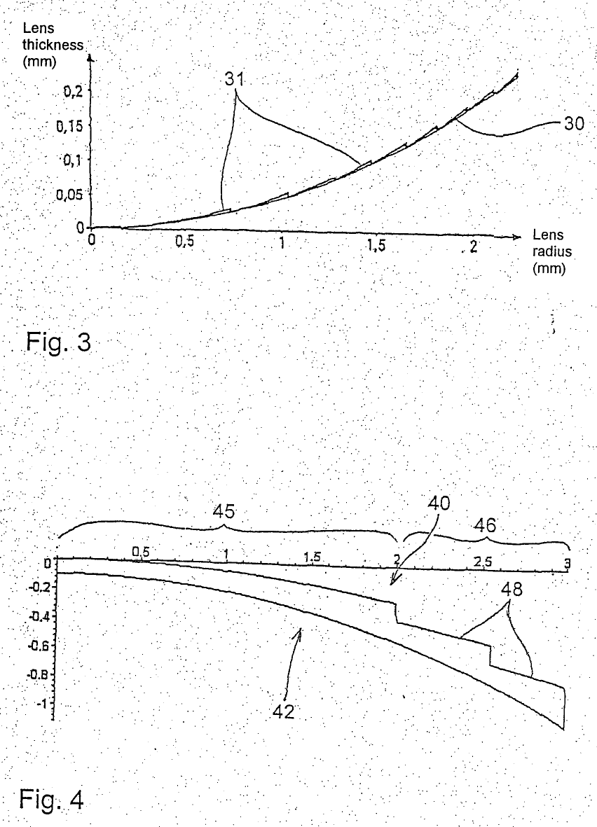

FIG. 3 shows a view on an enlarged scale of a portion of the lens front to explain an additional diffractive fine structure,

FIG. 4 shows a sectional view through one half of a further embodiment of a lens body according to the invention,

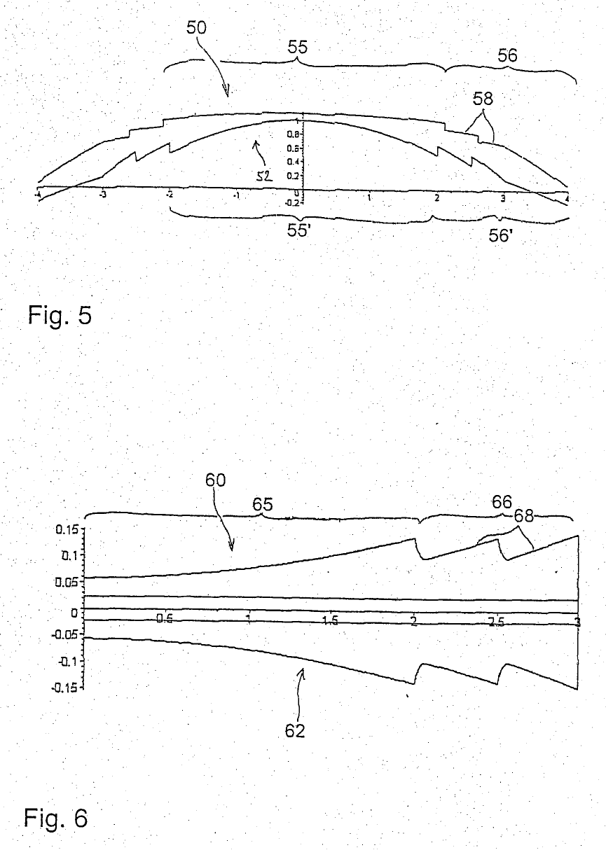

FIG. 5 shows a sectional view through a further embodiment of the intraocular lens according to the invention,

FIG. 6 shows a sectional view through one half of a further embodiment of a lens body according to the invention,

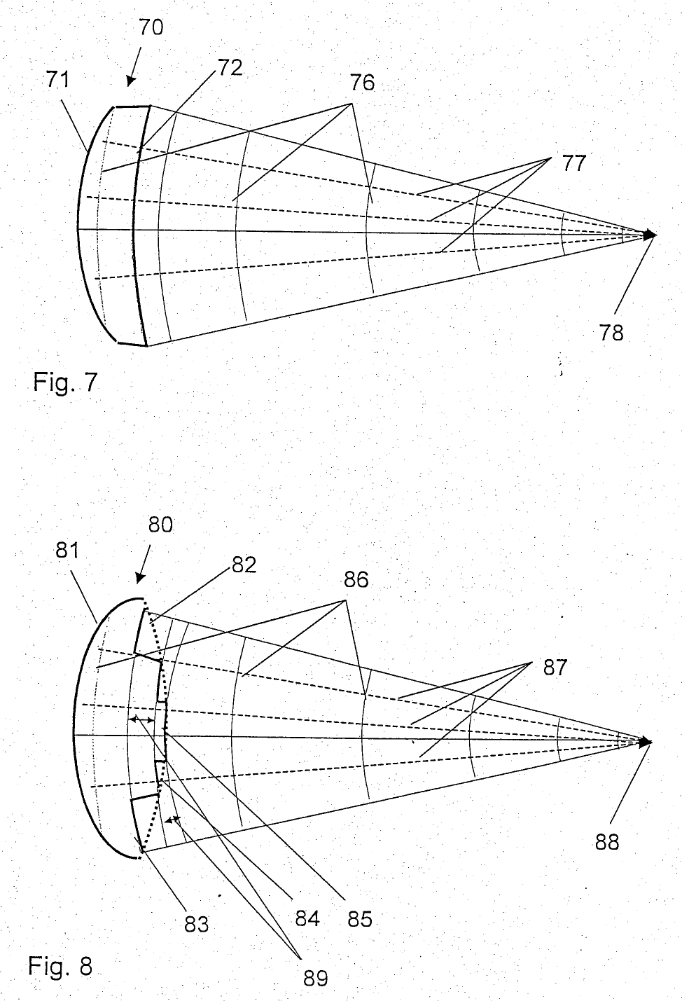

FIG. 7 shows a lens according to the invention with a purely refractive rear face, and

FIG. 8 shows a view on an enlarged scale of a portion of the lens front to explain an additional diffractive fine structure.

The imaging conditions in FIG. 1 which are described hereinafter are produced from a simulation model constructed by means of an optical design tool. FIG. 1 shows a diagrammatically illustrated portion of an eye with an also diagrammatically illustrated view of the intraocular lens 12 according to the invention. An axis-parallel beam 14 emanating from an infinitely far away article firstly passes through the cornea of the eye, which is shown here diagrammatically as an effective face 10, and the anterior chamber of the eye, and is already refracted on its way to the intraocular lens by virtue of different refractive indices. Upon reaching the intraocular lens the axis-parallel beam is therefore already refracted into an entry beam with a curved wave front. That wave front generally involves an approximately spherical curvature but it can also be greatly aspherically curved. That fact can be suitably taken into consideration in the design of the intraocular lens 12, that is to say when calculating the imaging properties thereof and thus the curvature of the front face, inter alia in dependence on the refractive index thereof. The beam 14 which impinges on the front face of the intraocular lens 12 is refracted and/or diffracted by virtue of the imaging properties thereof into an outgoing beam which is focused on to the retina, depending on whether this is a lens with refractive, diffractive (Fresnel) or mixed refractive-diffractive component. In accordance with the invention the rear face of the intraocular lens is of a spherical curvature configuration whose radius depends solely on the spacing of the implanted intraocular lens with respect to the retina at the focal point 18, that is to say on the optical axis or the central axis of the lens. By virtue thereof the curvature of the rear face corresponds to the curvature of the wave fronts of the outgoing beam 16 which is not once again diffracted and/or refracted there.

The half-section shown in FIG. 2 of an intraocular lens according to the invention shows the positively refractive front face 20 thereof, which is subdivided into a central lens region 25 with a purely refractive property and an annular lens region 26 which concentrically surrounds same, with superposed, positively diffractive, annular zones 28. The rear face 22 has throughout a spherical curvature configuration while the front face 20 overall is of an aspherical curvature configuration. The surfaces of the two concentric annular zones 28 provided in the annular lens region 26 are respectively displaced relative to each other in a sawtooth-like relationship in the direction of the optical axis in such a way that at the transition from the central lens region 25 to the inner annular zone and at the transition from the inner to the outer annular zone the difference in path length of the ray path is an integral multiple of the design wavelength. The design wavelength is usually 550 nm, and thus is in the range of green light. By virtue of that measure the thickness of the intraocular lens can be reduced so that it can be folded and is thus easier to implant without the imaging properties being substantially altered. In the illustrated embodiment the central lens region is approximately of a diameter of 4 mm while the annular lens region 26 disposed in the edge region of the optical lens part is of a width of 1 nm in this case.

By virtue of the fact that the spherical form of the rear face 22 means that refraction of the intraocular lens for axis-parallel rays along that face is 0, the position of the focus does not change for those rays in dependence on the refractive index of the medium behind the intraocular lens. Only the imaging scale can change by the following value:

$$m = \frac{{n}_{tamponade}}{{n}_{vitreoushumor}} .$$

If for example the vitreous humor of the eye is firstly replaced after a vitrectomy by silicone oil (n_{silicone oil}=1.43) and later the silicone oil is replaced by water (n=1.336), the consequence of that is that the patient sees an image which is increased in size by 7%.

In addition to the refractive component of the light passing through the lens, which is formed both in the central lens region 25 and also in the peripheral annular lens region 26, a determinable proportion of the light can be diffracted in the case of an intraocular lens as shown in FIG. 3 by means of a diffractive fine structure 31, here only on the front face 30 of the intraocular lens. While the focus of the refractive component is set for example for vision in the far range, a second focus can be formed by a design of the diffractive fine structure 31, which is matched to the conditions in the eye of the patient, and that second focus permits sharp vision in the near range. In that case, reference is made to a bifocal lens. The fine structure is preferably in the form of a diffractive pattern and is in the form of annular fine structure elements which generally are in concentric relationship with the optical axis and are of a sawtooth-shaped configuration displaced in the direction of the optical axis.

FIG. 3 shows the substantially spherical component of the section curve of the front face in the central lens region. Starting from the substantially spherical basic curve 30 the annular diffractive zones are of depths of between 1.0 μm and 5 μm. Those depths of the diffractive fine structures of bifocal lenses afford a difference in path length between adjacent zones of a fraction, for example 0.4 and 0.6 respectively, of the design wavelength, typically once again green light at a wavelength of 550 nm.

The additional diffractive fine structure pattern is preferably provided in the central lens region. It can however also extend over the annular lens region 26 and superpose the annular zones in that region. A further embodiment of an intraocular lens according to the invention is shown in FIG. 4 once again as a half-section. This involves a lens with positively refractive (convex) front face 40 which in addition to the central lens region 45 with a purely refractive property is subdivided into an annular edge region 56 concentrically surrounding same, with superposed, negatively diffractive, annular zones 48. The rear face 42 is once again throughout of a spherical curvature configuration. With that lens the effective local curvature of the front face is less than the macroscopic curvature of the entire lens front, both in the central lens region 45 and also in the individual annular zones 48.

FIG. 5 shows yet another embodiment of an intraocular lens according to the invention. In this example also the front face 50 is positively refractive (convex) with a purely refractive central lens region 55 and an edge region 56 with superposed negatively diffractive, concentric, annular zones 58. Here however the concave rear face 42 is in turn subdivided into a central, purely refractive region 55′ and an annular edge region 56′ with superposed diffractive structures 58′ of concentric rings of a sawtooth configuration. That provides that the effective local curvature of the rear face 52 in the central lens region 55′ and also in the individual annular zones 56′ is greater than the macroscopic curvature thereof. That overall provides a very thin lens.

The embodiment of FIG. 6, only shown in a half-section, for the first time has a concave front face 60 with an edge region 66 with superposed, negatively diffractive, concentric, annular zones 68. Here the effective local curvature of the front face 60, both in the central lens region 65 and also in the individual annular zones 68, is greater that the macroscopic curvature of the overall lens front. The convex rear face 62 is of a configuration as in the preceding embodiment of FIG. 5 whereby overall a comparatively thin lens design is achieved.

The embodiment of the intraocular lens 70 according to the invention as shown in FIG. 7 has purely refractive boundary faces, that is to say a front face 71 and a rear face 72 without diffractive structures. A refractive action is achieved however only by the front face 71 which is remote from the retina. The curvature of the rear face corresponds to the curvature of the wave fronts 76 at the rearward lens edge, that is to say at the transition from the lens to the vitreous humor medium. The rays 77 which are perpendicular to the wave fronts are at a right angle to the rear face and are accordingly not refracted. The spacing of the focus 78 with respect to the intraocular lens and thus the overall optical system therefore remains unchanged irrespective of the refractive index of the medium therebehind.

FIG. 8 shows an intraocular lens 80 according to the invention with a purely refractive front face 81 and a diffractive rear face. The macroscopic form of the rear face, illustrated by the envelope curve 83 in broken line, is convex while the diffractively acting face portions 82, 83, 84 are of a concave form. The curvatures of the diffractive face portions 82, 83, 84 correspond to the curvatures of the wave fronts 86 at the respective location of the face portions, that is to say at the transition from the lens to the vitreous humor medium. A refractive action is therefore again produced only by the front face 81.

It is possible to see steps between adjacent diffractive face portions 82, 83, 84 or zones. The geometrical heights of the steps are so selected that the optical difference 89 in path length between two diffractive face portions 82, 83, 84 is an integral multiple N of the design wavelength. In that case, it is possible to embody from one face portion to the next, different step heights which correspond to differences in path length of various integral multiples of the design wavelength. It is also possible to provide steps in various directions, that is to say of differing signs.

In the case of an integral multiple of preferably N>10 and a refractive index for the lens of 1.46 and a refractive index for the vitreous humor or chamber water of 1.336, the basic equation:

$$h = N \cdot \frac{λ}{{n}_{lens} - {n}_{surroundingmedium}}$$

gives for example a height of about N·8·λ, wherein N is an integral number nₗₑₙₛ and n_{surrounding medium} are the refractive indices and λ, is the design wavelength. As it will be appreciated that there are different values for h for media with different refractive indices in the vitreous humor chamber with the same N, the height h is preferably so selected that it nonetheless represents for both media a respectively different integral multiple N and M respectively in respect of the difference in path length. The geometrical heights of the steps h are accordingly afforded by:

$$h = \frac{N \cdot λ}{{n}_{lens} - {n}_{surroundingmediium\_ 1}} = \frac{M \cdot λ}{{n}_{lens} - {n}_{surroundingmedium\_ 2}} .$$

wherein N and M are integral numbers and nₗₑₙₛ, n_{surrounding medium}_{_{—}}₁ and n_{surrounding medium}_{_{—}}₂ are the refractive indices of the intraocular lens and two different given tamponades in the vitreous humor chamber of the eye.

In accordance with the invention the curve radii of the individual face portions change with the spacing of the face portions with respect to the focal point 88.

The edges of the steps between two adjacent diffractive face portions preferably meet the face portions at a right angle, and thus extend in the beam direction, in order to reduce scatter light at the edges.

Overall it is possible in that way to produce thin intraocular lenses of different refractive power, which all have a rear face with an optically effective curvature which corresponds to the curvature of the wave front incident from the respective front face. Basically in that respect, from one lens design to another lens design, both the thickness of the lens, the relationships of the central lens region to the annular lens region and also the number and height of the annular zones can be varied inter alia in dependence on the refractive index of the lens material, in accordance with the respectively desired refractive power of the lens. The sawtooth-shaped ring structure can also be provided additionally or exclusively at the rear face of the intraocular lens. Equally, a respectively different lens material with differing refractive indices can be used for the central lens region and the annular lens region or also the individual annular zones, thus affording still further possible variations in the lens design.

1. An intraocular lens comprising an optical lens part which has a rear face (12, 22, 42, 52, 62) which can be towards the retina, wherein at least one optically effective face portion of the rear face has an at least approximately spherical curvature configuration, whose radius of curvature corresponds to the spacing of the face portion with respect to the retina in the region of greatest visual acuity.

2. An intraocular lens as set forth in claim 1 characterised in that the radius of curvature of the rearward face portion is between 12 mm and 30 mm.

3. An intraocular lens as set forth in claim 1 characterised in that the radius of curvature is preferably between 13 mm and 23 mm.

4. An intraocular lens as set forth in one of the preceding claims characterised in that a first optically effective face portion with an at least approximately spherical curvature configuration is provided in a central, purely refractive lens region (25, 45, 55, 65) of the rear face.

5. An intraocular lens as set forth in claim 4 characterised in that at least one second optically effective face portion with an at least approximately spherical curvature configuration is provided in a diffractive lens region (26, 46, 56, 66) of the rear face, surrounding the central lens region (25, 45, 55, 65), concentrically adjoining the first optically effective face portion, wherein the radius of curvature of the second optically effective face portion corresponds to the spacing thereof with respect to the retina in the region of greatest visual acuity.

6. An intraocular lens as set forth in claim 4 or claim 5 characterised in that further optically effective face portions with an at least approximately spherical curvature configuration are provided in the annular diffractive lens region (26, 46, 56, 66) concentrically adjoining the second optically effective face portion and also each other, wherein the radius of curvature of the further optically effective face portions respectively corresponds to the spacing thereof with respect to the retina in the region of greatest visual acuity.

7. An intraocular lens as set forth in claim 6 characterised in that the difference in path length of the ray path in the transition of respectively adjacent optically effective face portions is an integral multiple of the design wavelength.

8. An intraocular lens as set forth in one of claims 5 through 7 characterised in that the geometrical step height h between respectively adjacent optically effective face portions in parallel relationship with the ray path satisfies the following equation:
h·(n_{IOL}−n_{surrounding medium}_{_{—}}₁)·N=h·(n_{IOL}−n_{surrounding medium}_{_{—}}₂)·M,
wherein N and M are whole numbers and n_{IOL}, n_{surrounding medium:}_{_{—}}₁ and n_{surrounding medium:}_{_{—}}₂ are the refractive indices of the intraocular lens and two different tamponades or substitution media in the vitreous humor chamber of the eye.

9. An intraocular lens as set forth in one of claims 5 through 8 characterised in that the optically effective face portions (28, 48, 58, 68) are of a sawtooth configuration.

10. An intraocular lens as set forth in one of claims 4 through 9 characterised in that the central lens region (25, 45, 55, 65) is of a diameter of about 4 mm.

11. An intraocular lens as set forth in one of the preceding claims characterised in that a diffractive fine structure (31) extends at least portion-wise over the front face to form a multifocal lens.

12. An intraocular lens as set forth in claim 11 characterised in that the diffractive fine structure (31) is provided in the central lens region (25, 45, 55, 65, 55′) of the front face (10, 20, 30, 40, 50, 60).

13. An intraocular lens comprising an optical lens part which has a front face (10, 20, 30, 40, 50, 60) which can be turned away from the retina and an oppositely disposed rear face (12, 22, 42, 52, 62), wherein the rear face has an optically effective curvature which corresponds to the curvature of a wave front incident from the front face (10, 20, 30, 40, 50, 60) on the rear face (12, 22, 42, 52, 62).

14. An intraocular lens as set forth in claim 13 characterised in that the optically effective curvature is spherical.

15. An intraocular lens as set forth in claim 14 characterised in that the rear face (12, 22, 42, 52, 62) has an at least portion-wise spherical curvature configuration whose radius of curvature at the face apex corresponds to the spacing with respect to the retina on the optical central axis.

16. An intraocular lens as set forth in claim 15 characterised in that the radius of curvature is between 12 mm and 30 mm.

17. An intraocular lens as set forth in claim 16 characterised in that the radius of curvature is between 13 mm and 23 mm.

18. An intraocular lens comprising an optical lens part which has a rear face (12, 22) which can be towards the retina, with an at least portion-wise spherical curvature configuration whose radius of curvature corresponds to the spacing of the rear face (12, 22) with respect to the retina on the optical central axis.

19. An intraocular lens as set forth in one of claims 13 through 18 characterised in that the optical lens part has a central lens region (25, 45, 55, 65) and an annular lens region (26, 46, 56, 66) surrounding same, with a common focus (18), wherein the annular lens region (26, 46, 56, 66) has at least one first concentrically annular zone (28, 48, 58, 68), wherein the difference in path length of the ray path at the transition from the central lens region (25, 45, 55, 65) to the first annular zone and at the transition of respectively adjacent annular zones (28, 48, 58, 68) is an integral multiple of n>=2 of the design wavelength.

20. An intraocular lens as set forth in claim 19 characterised in that the annular zones (28, 48, 58, 68) are of a sawtooth configuration.

21. An intraocular lens as set forth in one of claims 19 and 20 characterised in that the annular zones (28, 48, 58, 68) are provided on the front face (10, 20, 30, 40, 50, 60) of the optical lens part.

22. An intraocular lens as set forth in one of the preceding claims characterised in that the optical lens part is of an aspherical curvature configuration along the front face (10, 20, 30).

23. An intraocular lens as set forth in one of the preceding claims characterised in that the central lens region (25, 45, 55, 65) is of a diameter of about 4 mm.

24. An intraocular lens as set forth in one of the preceding claims characterised in that a diffractive fine structure (31) extends at least portion-wise over the optical lens part to form a multifocal lens.

25. An intraocular lens as set forth in claim 24 characterised in that the diffractive fine structure (31) is provided in the central lens region (25, 45, 55, 65, 55′) of the front face (10, 20, 30, 40, 50, 60) and/or rear face (12, 22, 42, 52, 62).

26. An intraocular lens as set forth in claim 24 or claim 25 characterised in that the diffractive fine structure (31) has fine structure zones (31), wherein the difference in path length of the ray path at the transition of respectively adjacent fine structure zones (31) is alternately between 0.5 and 0.8 times and between 0.5 and 0.2 times respectively and is preferably 0.4 times and 0.6 times respectively the design wavelength.
